(19) United States
(12) Patent Application Publication
(10) Pub. No.: US 2025/0074855 A1
(43) Pub. Date: Mar. 6, 2025

(54) STEREOSELECTIVE PREPARATION OF TRANS HALO CYCLOBUTANE

(71) Applicant: AMGEN INC., Thousand Oaks, CA (US)

(72) Inventors: Adrian ORTIZ, Oak Park, CA (US); Athimoolam ARUNACHALAMPILLAI, Hosur Tamilnadu (IN)

(73) Assignee: AMGEN INC., Thousand Oaks, CA (US)

(21) Appl. No.: 18/291,294

(22) PCT Filed: Aug. 17, 2022
(86) PCT No.: PCT/US2022/040666

§ 371 Date: Jan. 23, 2024

Related U.S. Application Data
(60) Provisional application No. 63/234,935, filed on Aug. 19, 2021.

(51) Int. Cl. C07C 51/09; C07C 17/18; C07C 51/42

(52) U.S. Cl. CPC C07C 51/09; C07C 17/18; C07C 51/42; C07C 2531/12

(57) ABSTRACT

The present invention relates to stereoselective process for the preparation of a compound having formula (2) and (1) wherein X is defined in the specification.

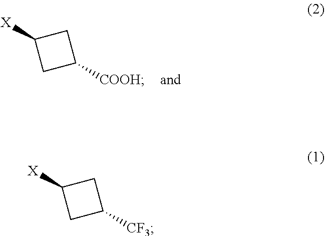

### FIELD OF THE INVENTION

The present invention relates to novel stereoselective processes for preparation of trans-halo-carboxy cyclobutyl and trans-halo-CF₃ cyclobutyl compounds having a high enantiomeric ratio.

### BACKGROUND OF THE INVENTION

The present application relates to novel stereoselective processes for preparation of a halo cyclobutyl compound of formula (1):

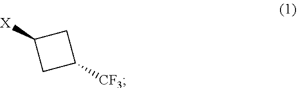

wherein: X is as defined below, or a pharmaceutically acceptable salt thereof. Preferably, the compound is a trans-halo-CF₃ homocyclic compound, more preferably trans-halo-CF₃ cyclobutane, trans-halo-CF₃ cyclopentane, or trans-halo-CF₃ cyclohexane. A most preferred compound is trans-Br—CF₃ cyclobutane (Compound 1a):

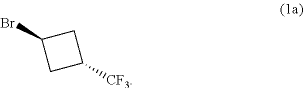

In a known route, compound (1a) was prepared from starting material compound keto ester

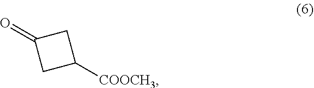

which is likely prepared through a 2+2 cycloaddition reaction:

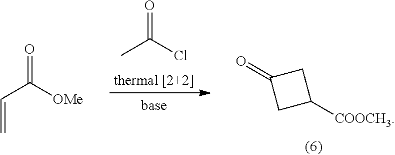

The keto ester starting material was converted to racemic Br—COOH cyclobutane, which was then separated through a column chromatography to the trans-Br—COOH cyclobutene and cis-Br—COOH cyclobutene isomers.

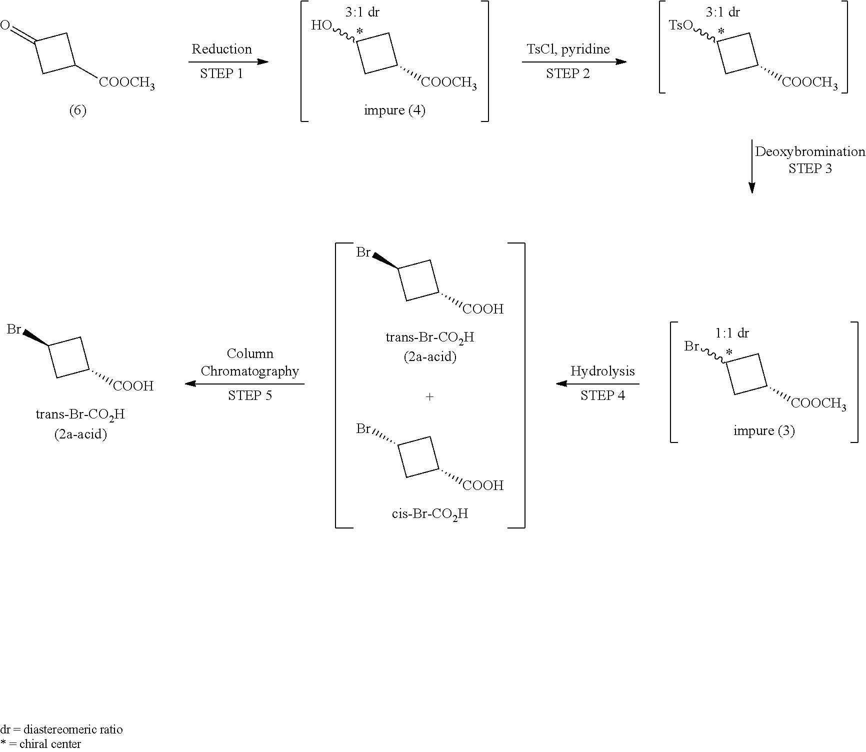

After column separation, the desired trans-Br—COOH cyclobutane isomer was then isolated and converted to the trans-Br—CF₃ cyclobutane as follows:

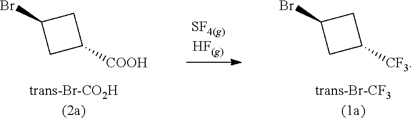 [SMILES: O=C(O)[C@H]1C[C@H](Br)C1 ; FC(F)(F)[C@H]1C[C@H](Br)C1]

See Bugera, M. et. al.; Deoxofluorination of Aliphatic Carboxylic Acids: A Route to Trifluoromethyl-Substituted Derivative. J. Org. Chem. 2019, 84, 16105-16115.

The above known preparation of compound (1a) production lacks selectivity resulting in chromatographic separation of cis/trans isomers in a late stage intermediate with low yield. The conversion of —CO₂H moiety to the —CF₃ moiety requires unique facilities and trained operators to safely handle the required highly hazardous reagents, which leads to extreme prices and lead times for production. It is not surprising that compound (1a) is available only as a custom order building block compound CAS #2306248-65-5 with long lead times. With only limited suppliers available, e.g., Enamine Ltd, Ukraine, compound (1a) sells for about US$80,000 per kg.

There is therefore an unmet need to prepare compound (1a) in a more cost effective and efficient way.

The present inventors have developed a novel stereospecific large scale synthetic technology to prepare compound (1) as follows:

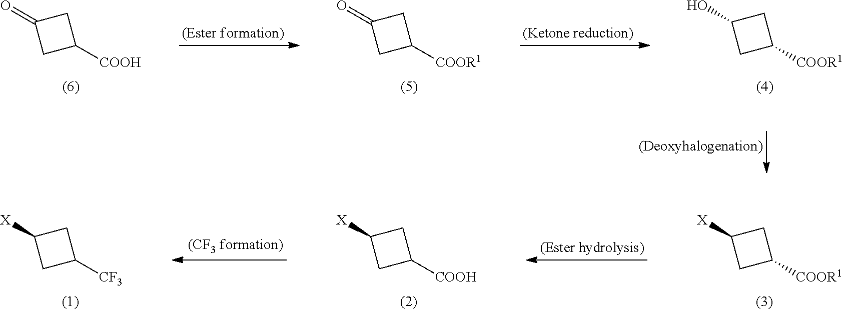

Specifically, the present inventors invented a process to synthesize compound (1a) by using the above process, which enable them to control timelines and produce supply source of compound (1a). In each step of the process of the invention, the stereochemistry of the product is carefully controlled. For example, in the Ketone reduction process, the keto-ester cyclobutyl (5) starting material is converted to cis hydroxy-ester cyclobutyl (4) product at a 95:5 diastereomeric ratio, which is then converted to trans halo-ester cyclobutyl (3) product in the subsequent fully stereospecific Deoxyhalogenation process at very high diastereomeric ratio. This 95:5 diastereomeric ratio Ketone reduction step of the invention provides advantages over the 3:1 diastereomeric ratio Ketone reduction step of the known process. The trans compound (3) then retains its trans configuration throughout the rest of the remaining processes of the invention at a high diastereomeric ratio, i.e., the Ester hydrolysis process to form trans Compound (2) and the CF₃ formation process to form compound (1). Naturally, such highly stereospecific processes of the inventions are desirable in the art, as they provide higher yields of the desired stereochemical products.

The present inventors have further created a solution to further improve the stereoselectivity of the Ketone Reduction process from 95:5 diastereomeric ratio to achieving a 99.8:0.2 diastereomeric ratio of the desired cis hydroxy-ester cyclobutyl (4) product through development of a biocatalytic reduction via ketoreductase enzyme (KRED) of ester ketone cyclobutane compounds. Such stereospecific enzymatic process coupled with the subsequent fully stereospecific Deoxybromination process eliminates chromatography which results in higher yields and greater faster throughput to prepare compound (1), specifically compound (1a).

Overall, the present inventors have invented a new process for the manufacture of compounds of formula (1) and (2) which offer a number of relevant benefits as compared to processes known in the art in the following ways: (a) A highly stereoselective reaction; (b) Subsequent purification using chiral chromatography is eliminated; (c) Improved overall yield; (d) More efficient, greener and less costly overall reaction; and (e) Scalable reactions.

### SUMMARY OF THE INVENTION

A first aspect of the present invention provides a stereoselective, improved, safer, cost effective and scalable process for the preparation of a compound having formula (2):

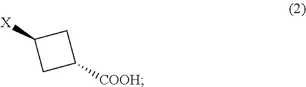

wherein X is halo; or a pharmaceutically acceptable salt thereof.

In embodiment 1, the present invention provides a stereoselective process for the preparation of a compound having formula (2):

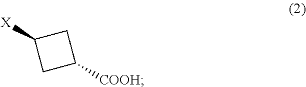

wherein X is halo; comprising
(a) contacting a compound of formula (3):

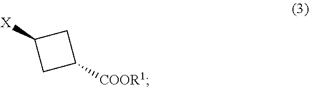

wherein X is as defined above in compound (2); and COOR¹ is an ester group; with an ester hydrolyzing agent in a solvent; to form said compound (2); and optionally followed by a process of purifying said compound (2) by
(a1) contacting said compound (2) with a base in a solvent to form a salt of compound (2); and
(a2) contacting said salt of compound (2) with an acid in a solvent at a low temperature to form a purified form of compound (2). Preferably, the process of purifying said compound (2) is performed.

In embodiment 1a, the present invention provides the process according to embodiment Error! Reference source not found., further comprising preparing said compound (3) comprising:
(b) contacting a compound of formula (4):

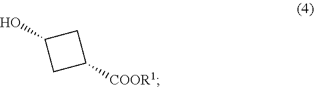

wherein said R¹ is as defined above in compound (3); with a deoxyhalogenating agent in an organic solvent, optionally at a low temperature, to form said compound (3).

In embodiment 1b, the present invention provides the process according to embodiments Error! Reference source not found. and 1a, further comprising preparing said compound (4) comprising: contacting a compound of formula (5):

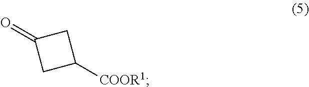

wherein said R¹ is as defined above in compound (4) with;
(c1) a metal catalyst in an organic solvent at a low temperature; or
(c2) a biocatalytic agent in an organic solvent and in the presence of a buffer solution; to form said compound (4).

Preferably, the process (c1) provides a higher than 90% stereomeric selectivity; more preferably higher than 95% stereomeric selectivity; for the cis configuration of the compound (4) product.

Preferably, the process (c2) provides a higher than 95% stereomeric selectivity; more preferably higher than 99% stereomeric selectivity; for the cis configuration of the compound (4) product.

In embodiment 1c, the present invention provides the process according to embodiments Error! Reference source not found., 1a, and 1b, further comprising preparing compound (5) comprising:
(d) contacting a compound of formula (6):

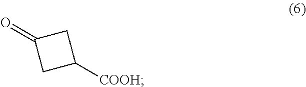

with an R¹ agent, in the presence of a base; wherein R¹ is as defined above in compound (5); in an organic solvent to form said compound (5).

Another aspect of the present invention provides a stereoselective, improved, safer, cost effective and scalable process for the preparation of a compound having of formula (1) from said compound of formula (2).

In this aspect of the invention, in embodiment 2, the present invention provides the process according to embodiment 1, including any of sub-embodiments 1a-1c, further comprising preparing a compound of formula (1):

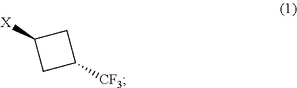

wherein said X is as defined above in compound (2); comprising: contacting said compound (2) with a trifluoromethylating agent in an organic solvent to form said compound of formula (1).

In embodiment 3, the present invention provides the process according to embodiment 1, 1a-1c, or 2, wherein X is bromo or iodo. Preferably, X is bromo.

In embodiment 4, the present invention provides the process according to embodiment 1, 1a-1c, 2, or 3, wherein R¹ is (C₁-C₆)alkyl, phenyl, or benzyl. Preferably, R¹ is benzyl.

In embodiment 5, the present invention provides the process according to embodiment 1, 1a-1c, 2, 3, or 4, wherein in (a), said solvent is a mixture of methyl-THF and water.

In embodiment 5a, in (a), said ester hydrolyzing agent is an alkali metal hydroxide or a lipase enzyme.

In embodiment 5b, in (a), said ester hydrolyzing agent is alkali metal hydroxide and said solvent is a mixture of methyl-THF and water. In this embodiment, preferably, the ester hydrolyzing agent is sodium hydroxide, potassium hydroxide, or lithium hydroxide. In this embodiment, more preferably, said ester hydrolyzing agent is lithium hydroxide or sodium hydroxide.

In embodiment 5c, in (a), said ester hydrolyzing agent is a lipase enzyme and said solvent is acetone or isopropyl alcohol. In this embodiment, preferably, said ester hydrolyzing agent is amano lipase enzyme.

In embodiment 6, the present invention provides the process according to embodiment 1, 1a-1c, 2, 3, 4, or 5, wherein in (a1), said base is a primary, secondary, or tertiary amine base. Preferably, said base is tert-butyl amine and said salt of compound (2) has a formula:

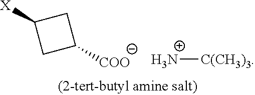

In this embodiment, said salt of compound (2) is a non-hygroscopic salt.

In embodiment 6a, in (a1), said solvent is a mixture of n-heptane and MTBE.

In embodiment 7, the present invention provides the process according to embodiment 1, 1a-1c, 2, 3, 4, 5, 5a, 5b, 5c, 6, or 6a, wherein in (a2), said acid is sulphuric acid, phosphoric acid, or acid halide selected from HCl or HBr. In this embodiment, preferably said acid is halide acid selected from HCl, or HBr.

In embodiment 7a, in (a2), said solvent is water.

In embodiment 7b, in (a2), said at a low temperature is between 0° C. to −5° C.; or 0° C.

In embodiment 8, the present invention provides the process according to embodiment 1, 1a-1c, 2, 3, 4, 5, 5a, 5b, 5c, 6, 6a, 7, 7a, or 7b, wherein in (b), said deoxyhalogenating agent is triphenyl phosphite in the presence of NBS; or triphenyl phosphine in the presence of NBS. Preferably, the deoxyhalogenating agent is triphenyl phosphite in the presence of NBS.

In embodiment 8a, in (b), said organic solvent is DMF, acetonitrile, toluene, or dichloromethane.

In embodiment 8b, in (b), said low temperature is below 0° C.; or between −10° C. to −5° C.

In embodiment 9, the present invention provides the process according to embodiment 1, 1a-1c, 2, 3, 4, 5, 5a, 5b, 5c, 6, 6a, 7, 7a, 8, 8a, or 8b, wherein in (c1), said metal catalyst is a metal hydride.

In embodiment 9a, in (c1), said metal hydride is sodium borohydride, lithium aluminum hydride, LiAl(OtBu)₃ or diisobutylaluminium hydride (DIBAL-H). Preferably, said metal hydride is LiAl(OtBu)₃.

In embodiment 9b, in (c1), said solvent is THF, acetonitrile, toluene, dichloromethane, or (C₁-C₈)alcohol selected from methanol, ethanol, or isopropyl alcohol, or any mixtures thereof.

In embodiment 9c, in (c1), said low temperature is between −78° C. to −5° C.; or between −10° C. to −5° C.; or 0° C. Preferably, said low temperature is 0° C.

In embodiment 9d, in (c1), the metal hydride is DIBAL-H and said low temperature is −78° C.

In embodiment 9e, in (c1), the metal hydride is LiAl(OtBu)₃ and said low temperature is between −10° C. to −5° C.; or 0° C.

In embodiment 10, the present invention provides the process according to embodiment 1, 1a-1c, 2, 3, 4, 5, 5a, 5b, 5c, 6, 6a, 7, 7a, 8, 8a, 8b, 9, 9a, 9b, 9c, 9d, or 9e wherein in (c2), said biocatalytic agent is a ketoreductase enzyme.

In embodiment 10a, in (c2), said ketoreductase enzyme is KRED, such as KRED-P3-G09, in the presence of a co-factor, including NADP⁺.

In embodiment 10b, in (c2), said solvent is (C₁-C₈)alcohol, or a mixture of water and (C₁-C₈)alcohol. Preferably, said solvent is methanol, ethanol, isopropanol, or any mixtures thereof.

In embodiment 10c, in (c2), said buffer is triethanol amine buffer, potassium phosphate buffer, sodium phosphate buffer, potassium dihydrogen sulphate buffer, potassium sulphate buffer, sodium tetraethylborate buffer, or sodium tetraborate buffer. Preferably, said buffer is said buffer is triethanol amine buffer.

In embodiment 11, the present invention provides the process according to embodiment 1, 1a-1c, 2, 3, 4, 5, 5a, 5b, 5c, 6, 6a, 7, 7a, 8, 8a, 8b, 9, 9a, 9b, 9c, 9d, 9e, 10, 10a, 10b, and 10c, wherein in (d), said R¹ agent is (C₁-C₆)alkyl halide, phenyl halide, or benzyl halide.

In embodiment 11a, in (d), said R¹ agent is benzyl bromide.

In embodiment 11b, (d) is performed in the presence of a base, wherein said base is a bicarbonate, carbonate, or tri(C₁-C₆)alkylamine base. Preferably, the base is bicarbonate or carbonate base.

In embodiment 11c, in (d), said solvent is dichloromethane, DMF, THF, or acetonitrile. Preferably, said solvent is DMF.

In embodiment 11d, (d) is performed at room temperature.

In embodiment 12, the present invention provides the process according to embodiment 2, wherein said trifluoromethylating agent is sulfur tetrafluoride (SF₄), in the presence of hydrogen fluoride (HF), and optionally in the presence of a solvent.

In embodiment 12a, the present invention provides the process according to embodiment 2, wherein said trifluoromethylating agent is sulfur tetrafluoride (SF₄), in the presence of hydrogen fluoride (HF), and said process is performed in the presence of dichloromethane.

In embodiment 12b, the present invention provides the process according to embodiment 2, wherein said process is performed at a temperature between −78° C. to 30° C. Preferably, the temperature is maintained under 30° C.

### DETAILED DESCRIPTION

Unless otherwise stated, the following terms used in the specification and claims are defined for the purposes of this Application and have the following meaning:

“Alkali metal” refers to the chemical elements of Group 1 of the periodic table, i.e. lithium (Li), sodium (Na), potassium (K), rubidium (Rb), cesium (Cs), and francium (Fr). Particular examples of alkali metals are Li, Na and K, most particularly Na.

“(C_{α}-C_{β})Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Amino” or “Amine” means —NH₂.

“a primary, secondary, or tertiary amine” means an NH₃ group in which one, two, or three of its hydrogen atom(s) is/are substituted by a (C_{α}-C_{β})Alkyl group.

“Buffer” means an excipient, which stabilizes the pH of a process of chemical preparation. Suitable buffers are well known in the art and can be found in the literature. Particular pharmaceutically acceptable buffers comprise histidine-buffers, arginine-buffers, citrate-buffers, succinate-buffers, acetate buffers and phosphate-buffers. Independently from the buffer used, the pH can be adjusted with an acid or a base known in the art, e.g. hydrochloric acid, acetic acid, phosphoric acid, sulfuric acid and citric acid, sodium hydroxide and potassium hydroxide.

“Tri(C₁-C₆)alkylamine” means an amino group that is substituted by linear monovalent hydrocarbon radical of one to six carbon atoms or a branched monovalent hydrocarbon radical of three to six carbons. Examples include trimethylamine, triethylamine, and the like.

“(C_{α}-C_{β})Cycloalkyl” means a cyclic saturated monovalent hydrocarbon radical of three to ten carbon atoms wherein one or two carbon atoms may be replaced by an oxo group, e.g., cyclopropyl, cyclobutyl, cyclopentyl, or cyclohexyl, and the like.

“Cycloalkylalkyl” means a -(alkylene)-R radical where R is cycloalkyl as defined above; e.g., cyclopropylmethyl, cyclobutylmethyl, cyclopentylethyl, or cyclohexylmethyl, and the like.

“Carboxy” means —COOH or COO⁻M⁺; wherein M means a metal cation.

“Chiral center” means a carbon atom bonded to four nonidentical substituents. The term “chiral” denotes the ability of non-superimposability with the mirror image, while the term “achiral” refers to embodiments which are superimposable with their mirror image. Chiral molecules are optically active, i.e., the compounds containing them have the ability to rotate the plane of plane-polarized light.

“Diastereomer” means a stereoisomer with two or more centers of chirality and whose molecules are not mirror images of one another. Diastereomers have different physical properties, e.g. melting points, boiling points, spectral properties, and reactivities.

“Diastereomeric ratio” (dr) denotes the diastereomeric purity, which is the ratio of the percentage of one diastereoisomer in a mixture to that of the other diastereomer. Diastereomeric ratio can be calculated, for example from NMR spectra.

“Halo” or “Halogen” means fluoro, chloro, bromo, or iodo, preferably fluoro or chloro.

“Halo(C_{α}-C_{β})alkyl” means alkyl radical as defined above, which is substituted with one or more halogen atoms, preferably one to five halogen atoms, preferably fluorine or chlorine, including those substituted with different halogens, e.g., —CH₂Cl, —CF₃, —CHF₂, —CH₂CF₃, —CF₂CF₃, —CF(CH₃)₃, and the like. When the alkyl is substituted with only fluoro, it is referred to in this application as fluoroalkyl.

“Hydroxy(C_{α}-C_{β})alkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxy butyl, 3-hydroxy butyl, 4-hydroxybutyl, 2,3-dihydroxypropyl, 1-(hydroxymethyl)-2-hydroxyethyl, 2,3-dihydroxy butyl, 3,4-dihydroxy butyl and 2-(hydroxymethyl)-3-hydroxypropyl, preferably 2-hydroxyethyl, 2,3-dihydroxypropyl, and 1-(hydroxymethyl)-2-hydroxyethyl.

The present invention also includes protected derivatives of compounds of Formula (2) or formula (1). For example, when compounds of Formula (1) contain groups such as hydroxy, carboxy, thiol or any group containing a nitrogen atom(s), these groups can be protected with a suitable protecting group. A comprehensive list of suitable protective groups can be found in T. W. Greene, Protective Groups in Organic Synthesis, John Wiley & Sons, Inc. (1999), the disclosure of which is incorporated herein by reference in its entirety. The protected derivatives of compounds of Formula (1) can be prepared by methods well known in the art.

A “pharmaceutically acceptable salt” of a compound means a salt that is pharmaceutically acceptable and that possesses the desired pharmacological activity of the parent compound. Such salts include:
acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or
salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic. Additional information on suitable pharmaceutically acceptable salts can be found in Remington's Pharmaceutical Sciences, 17th ed., Mack Publishing Company, Easton, PA, 1985, which is incorporated herein by reference.

“Oxo, “keto”, or “carbonyl” means ═(O) group.

“Optional” or “optionally” means that the subsequently described event or circumstance may but need not occur, and that the description includes instances where the event or circumstance occurs and instances in which it does not. For example, “heterocyclyl group optionally substituted with an alkyl group” means that the alkyl may but need not be present, and the description includes situations where the heterocyclyl group is substituted with an alkyl group and situations where the heterocyclyl group is not substituted with alkyl.

“Stereoisomer” denotes a compound that possesses identical molecular connectivity and bond multiplicity, but which differs in the arrangement of its atoms in space.

### Generic Experimental Procedures

Compounds of Formula (1) and (2) wherein R¹ and X are as defined in the Summary of the Invention can be prepared as illustrated and described below:

### Step 1: Ester Formation

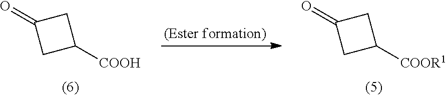

Treatment of a compound of formula (6) with an R¹ agent, wherein R¹ is as defined in the Summary of the Invention, provides a compound of formula (5) wherein R¹ is as defined in the Summary of the Invention. The reaction is carried out in a suitable organic solvent such as dichloromethane, DMF, THF, or acetonitrile, or the like, in the presence of a base such as bicarbonate, such as potassium bicarbonate, or carbonate base, such as potassium carbonate, or a tri(C₁-C₆)alkylamine base, such as triethylamine or trimethyl amine base, and takes place at a temperature between 25° C. to 30° C. The reaction takes between 12 h to 24 h, preferably 16 h. Suitable esterification R¹ agents include (C₁-C₆)alkyl halide, phenyl halide, or benzyl halide; or agents that form organic esters such as acetate, formate, or benzylate. Examples of esterification R¹ agents include benzyl bromide or benzyl chloride. Compounds of formula (6) and R¹ agents are either commercially available or can be readily prepared by methods well known in the art.

### Step 2: Ketone Reduction

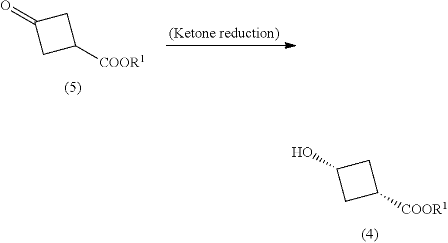

Treatment of a compound of formula (5) wherein R¹ is as defined in the Summary of the Invention with ketone reducing agent provides a compound of formula (4), wherein R¹ is as defined in the Summary of the Invention. The reaction can be carried out with a metal catalyst (Method A) or with a biocatalytic agent (Method B) as described below:

### Method A: Reduction With Metal Catalyst

Compound of formula (5) can be reacted with a metal catalyst ketone reducing agent in a suitable organic solvent such as THF, acetonitrile, toluene, dichloromethane, (C₁-C₈)alcohol, such as methanol, ethanol, or isopropyl alcohol, or any mixtures thereof, to provide a compound of formula (4). The reaction can take place at a temperature between −5° C. to 10° C., preferably between −5 to 5° C., most preferably about 0° C. The reaction takes between 3 h to 5 h, preferably about 4 h. Suitable metal catalyst ketone reducing agents include metal hydrides such as sodium borohydride, lithium aluminum hydride, LiAl(OtBu)₃ (formed from LiAlH₄ and tBuOH in situ) or Diisobutylaluminium hydride (DIBAL-H), and the like. Preferably, the metal hydride is LiAl(OtBu)₃, in which the reaction temperature can be controlled at non-cryogenic temperature. Further, LiAl(OtBu)₃ gives the best selectivity profile for reducing the ketone moiety to the hydroxy without reducing the ester moiety, thus minimizing side products. Alternatively, suitable metal catalyst ketone reducing agent includes DIBAL-H, where the reaction temperature is controlled at −78° C.

### Method B: Biocatalytic Reduction

Compound of formula (5) can be reacted with a biocatalytic ketone reducing agent, such as ketoreductase enzyme (KREDs), in a suitable organic solvent such as alcohol, such as methanol, ethanol, isopropanol, and the like; acetonitrile; and the like, to provide a compound of formula (4). The reaction takes place at a temperature between 25° C. to 30° C.; in the presence of a buffer and a co-catalyst or co-factor, such as NADP or NADPH, under nitrogen atmosphere. The reaction takes 12 h to 24 h, preferably 18 h. Suitable ketoreductases include KRED, preferably KRED-P3-G09. The reaction mechanism of the biocatalytic reduction of the present invention is generally depicted as follows:

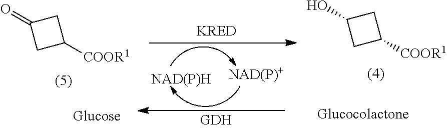

Various KRED enzymes were tested for compound (5) wherein R¹ is benzyl (i.e., compound (5a)), and the stereomeric excess results of the compound (4a) are tabulated below. KRED enzymes were purchased from CODEXIS, Inc. USA. LCAP was measured by liquid chromatography equipment.

| Reaction. | Name of keto-reductase | LCAP of |
|---|---|---|
| No. | (KRED) | cis-(4a) |
| 1. | KRED-P1-A04 | 97.0 |
| 2. | KRED-P1-A12 | 99.3 |
| 3. | KRED-P1-B02 | 83.6 |
| 4. | KRED-P1-B05 | 97.0 |
| 5. | KRED-P1-B10 | 99.0 |
| 6. | KRED-P1-B12 | 99.5 |
| 7. | KRED-P1-C01 | 84.3 |
| 8. | KRED-P1-H08 | 94.7 |
| 9. | KRED-P2-B02 | 75.7 |
| 10. | KRED-P2-C02 | 73.4 |
| 11. | KRED-P2-C11 | 98.8 |
| 12. | KRED-P2-D03 | 74.6 |
| 13. | KRED-P2-D11 | 99.6 |
| 14. | KRED-P2-D12 | 76.9 |
| 15. | KRED-P2-G03 | 99.2 |
| 16. | KRED-P2-H07 | 98.8 |
| 17. | KRED-P3-B03 | 99.8 |
| 18. | KRED-P3-G09 | 99.8 |
| 19. | KRED-P3-H12 | 99.9 |
| 20. | KRED-101 | 93.8 |
| 21. | KRED-119 | 99.9 |
| 22. | KRED-130 | 96.7 |
| 23. | KRED-NADH-101 | 98.9 |
| 24. | KRED-NADH-110 | 99.9 |

In one embodiment, the compound of formula (4) can be formed with a stereoselectivity for compound cis-4 having LCAP of at least 74%. In one aspect of this embodiment, the stereoselectivity can have LCAP of at least 75%. In a more particular aspect of this embodiment, the stereoselectivity can have LCAP of at least 93%. In a more particular aspect of this embodiment, the stereoselectivity can have LCAP of at least 99%.

The process of this invention requires the presence of a hydride source. The term “hydride source” refers to a compound or mixture that is capable of providing a hydride anion or a synthetic equivalent of a hydride anion. A hydride source may be used in catalytic or stoichiometric amounts. In case of the use of enzymes (KRED), additional co-factors are required in a catalytic amount. Thus, this combination of co-factor and KRED enzyme work together to regenerate a hydride from isopropyl alcohol and enable the reduction of the substrate.

A co-factor used with the ketoreductase enzyme in this process of the present invention is selected from Nicotinamide adenine dinucleotide (NAD), Nicotinamide adenine dinucleotide phosphate (NADP), Nicotinamide adenine dinucleotide hydrogen (NADH), and Nicotinamide adenine dinucleotide phosphate hydrogen (NADPH). The choice of co-factor may be based upon the presence or absence of a co-factor regeneration system. In embodiments where the hydride source does not comprise a co-factor regeneration system, the co-factor is in a stoichiometric amount and is a reduced co-factor which is therefore selected from NADH and NADPH for a hydride source. It is well known in the art, or information is available from the commercial supplier of the specific ketoreductase whether NADH or NADPH is the appropriate co-factor for a given ketoreductase. See, for example, https://www.codexis.com/wp-content/uploads/KRED-Product-Information.pdf. In this embodiment, the reduced co-factor is present in stoichiometric amounts as compared to the compound (5).

In another embodiment, the hydride source additionally comprises a co-factor regeneration system. The high cost of co-factors makes their use on a stoichiometric basis impractical. A low-cost co-factor regeneration system continually produces and regenerates the reduced form of the cofactor, requiring the co-factor to be present in only catalytic amounts. Moreover, the use of a co-factor regeneration system eliminates the need to use a reduced co-factor. The co-factor regeneration system produces the required reduced co-factor in situ. Accordingly, any cofactor or combinations of cofactors compatible with the chosen ketoreductase can be employed with a co-factor regeneration system. In this embodiment, therefore, NAD is interchangeable with NADH; and NADP is interchangeable with NADPH. Similarly, the designations “-NAD” and “-NADH”, and “-NADP” and “-NADPH”, respectively, are used interchangeably herein in conjunction with enzymes that use, respectively, NADH and NADPH as co-factors.

Suitable buffers include phosphate, triethanol amine, PIPES, BICINE, TES, TRIS, HEPES, TRICINE, CHES, or CAPS. Preferably, the buffer is triethanol amine.

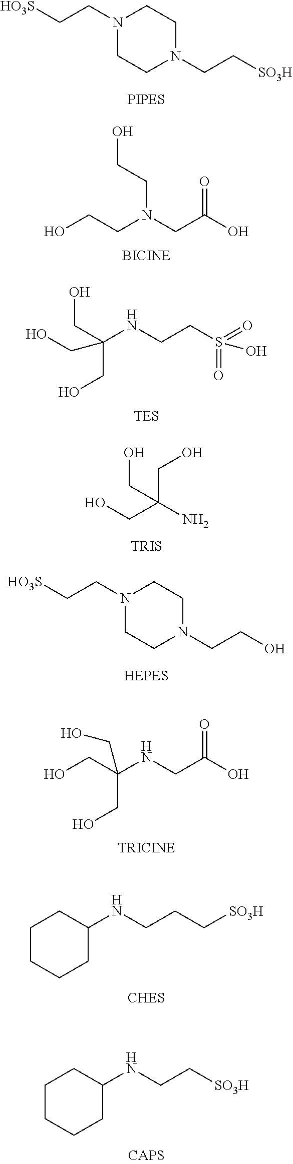

### Step 3: Deoxyhalogenation

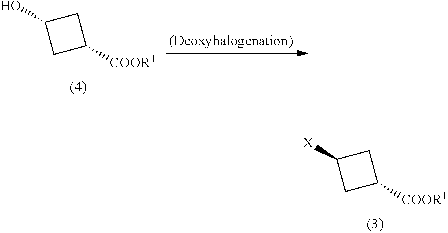

Treatment of a compound of formula (4) wherein R¹ is as defined in the Summary of the Invention, with a deoxyhalogenating agent provides a compound of formula (3), wherein X is halo and R¹ is as defined in the Summary of the Invention. The reaction is carried out in a suitable organic solvent such as DMF, acetonitrile, toluene, or dichloromethane, and the like, and takes place at between a temperature −10° C. to −5° C., or −10° C. to 0° C., preferably below 0° C. during the addition, and is then warmed to a temperature between 25° C. to 30° C. The reaction takes between 1 h to 2 h, preferably 1 h. Suitable deoxyhalogenating agents include triphenyl phosphite in the presence of NBS (preferred), or triphenyl phosphine in the presence of NBS.

### Ester Hydrolysis/Salt Formation

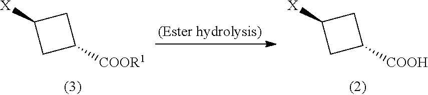

Treatment of a compound of formula (3) wherein X is halo, with an ester hydrolyzing agent provides a compound of formula (2). The reaction with an ester hydrolyzing agent is carried out in a suitable solvent such as methyl THF/water, or acetone, and the like, and takes place at a temperature between 25° C. to 30° C. Suitable ester hydrolyzing agents include alkali metal hydroxides, such as sodium hydroxide, potassium hydroxide, lithium hydroxide. Preferably, the alkali metal hydroxide is lithium hydroxide or sodium hydroxide. When alkali metal hydroxides are used, the suitable solvent is polar solvent such as a mixture of methyl-THF and water.

Alternatively, said ester hydrolyzing agent is a lipase enzyme. Suitable lipase enzymes include, for example, enzymes originated from a microorganism of Candida, such as Candida cylindracea and Candida rugosa, a microorganism of Chromobacterium chocolatum, pig liver and a thermophilic microorganism. Preferably, the lipase enzyme is Lipase PS Amano SD enzyme (AMANO ENZYME Inc., Nagoya, Japan), originated from Burkholderia cepacian, CAS #: 9001-62-1, LOT #: LPS1050808SD; in the presence of a buffer, such as phosphate buffer, and takes place at a temperature between 25° C. to 30° C., for 24 h. When a lipase enzyme is used, the suitable solvent is acetone or (C₁-C₆)alcohol, such as propanol or isopropyl alcohol.

Enzymatic resolution of the isomeric mixture may be achieved using techniques generally known in the art, including for example contacting an isomeric mixture with a suitable lipase enzyme, in order to selectively hydrolyze an ester moiety of the compound of Formula (3) or (3a), which is compound (3) wherein X is bromo and R¹ is benzyl. Due to the neutral pH conditions that are utilized with the lipase enzymes, the present inventors do not observe the by-products, such as diphenylphosphoric acid. Such by-product's physical properties are similar to the carboxylic acid product compound (2), which makes it difficult and tedious to remove without the use of lipase enzymes.

Preferably, the above ester hydrolysis step to form compound (2) followed by a process of purifying compound (2) is followed by purification of the compound (2) product. The purification process is done in two step reaction. The first step is reacting compound (2) with a base in a solvent to form a salt of compound (2); and then, in the second step, the salt of compound (2) is reacted with an acid in a solvent at a low temperature to form a purified version of compound (2).

In the first step, suitable base includes an amine base, such as a primary, secondary, or tertiary amine base. Preferred base is tert-butyl amine. Metal salts, such as sodium or calcium salts, can also be made to purify compound (2), however, Aminium salt is preferred compared to metal salts because the metal salts were found to be hygroscopic. Suitable solvent in the salt formation step includes n-heptane/MTBE, and the like. Each of the reactions takes between 12 h to 24 h, preferably 16 h.

In the second step, the salt of compound (2) obtained from the first step is reacted with an acid to form purified compound (2). Suitable acid includes sulphuric acid, phosphoric acid, or acid halide selected from HCl or HBr. Preferably, the acid is HCl. Suitable solvent includes water, lower alcohol, or mixture thereof. The reaction takes place at a temperature between −5° C. to 10° C., preferably between 0° C. to 5° C., most preferably at 0° C. The reaction takes between 1 h to 2 h, preferably 1 h.

### Trifluoromethylation

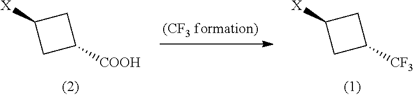

Treatment of a compound of formula (2), wherein X is halo, with a trifluoromethylating agent provides a compound of formula (1). The reaction is carried out in a suitable organic solvent such as dichloromethane, DMF, DMSO, and the like, and takes place at a temperature between −78° C. to 30° C. It is essential to maintain the reaction temperature to not exceed 30° C. because higher temperature was found to result in lower yield of compound (1a), which is compound (1) wherein X is bromo. The reaction takes between 12 h to 24 h, preferably 12 h. Suitable trifluoromethylatings include SF₄/HF reagent Those skilled in the art would understand that the above processes of the present invention can be performed in various orders and are not limited to the orders of the s described in the generic procedures above. The present inventors contemplate that the order of the reaction s of the present invention can vary. For example:

The invention will now be described in reference to the following specific Examples. These examples are not to be regarded as limiting the scope of the present invention, but shall only serve in an illustrative manner.

The following abbreviations are used throughout the description and appended claims, and they have the following meanings:
“DCM” means dichloromethane.
“DMSO” means dimethylsulfoxide
“EtOAc” means ethyl acetate
“h” means hour or hours
“HPLC” means high performance liquid chromatography
“IPA” means isopropyl alcohol.
“LCAP” means liquid chromatography area percent
“LCMS” means liquid chromatography mass spectrometry
“LiCl” means lithium chloride
“mins” means minutes
“MTBE” means methyl tertiary-butyl ether.
“rt” or “RT” means room temperature
“temp” means temperature
“t-bu” means tert-butyl

The chemicals used in the synthetic routes delineated herein include, for example, solvents, reagents, and catalysts. The methods described above may also additionally include steps, either before or after the steps described specifically herein, to add or remove suitable protecting groups in order to ultimately allow synthesis of the compounds. In addition, various synthetic s may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

All reagents, starting materials, and solvents (laboratory grade or anhydrous grade) were used as received. Purity was determined using reverse-phase HPLC. Chemical shifts (δ) for protons and carbon are reported in parts per million (ppm) referenced to tetramethylsilane (δ_{H}=0.00, δ_{C}=0.00) or to residual proton or carbon in the NMR solvent (CDCl₃: δ_{H}=7.26 ppm, δ_{C}=77.2 ppm.

### EXAMPLES: EXPERIMENTAL PROCEDURES

### Example 1: Synthesis of Benzyl 3-oxocyclobutane-1-carboxylate (5a)

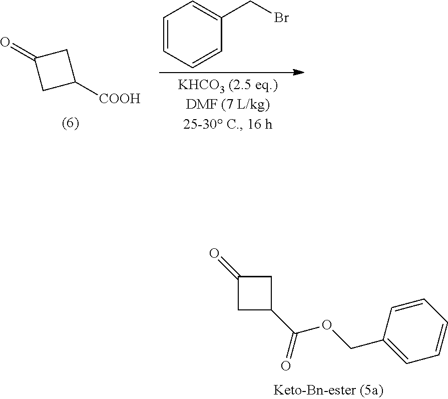 [SMILES: O=C1CC(C(=O)O)C1 ; BrCc1ccccc1 ; O=C1CC(C(=O)OCc2ccccc2)C1]

To a solution of 3-oxocyclobutane carboxylic acid (6), available in Sigma-Aldrich, (1.12 kg, 9.82 mol, 1.2 eq.) in DMF (9.8 L, 7 L/kg) in a glass reactor were added potassium bicarbonate (2.05 kg, 20.46 mol, 2.5 eq.) and benzyl bromide (1.4 kg, 8.19 mol, 1.0 eq.) under a nitrogen atmosphere at 25-30° C. The reaction mixture was stirred at 25° C. to 30° C. for 16 h. After the reaction was adjudged complete by HPLC, the reaction mixture was cooled to 5° C. to 10° C., quenched by the addition of water (14.0 L, 10 L/kg) and diluted with MTBE (14.0 L, 10 L/kg). The contents were warmed to 25° C. to 30° C. and the phases separated. The aqueous phase was extracted with MTBE (7.0 L, 5 L/kg) and the combined organic phase was washed with 20 wt % aq. LiCl solution (7.0 L, 5 L/kg) twice. The organic phase was distilled under vacuum at 35° C. to 40° C. to about 2 L. The resulting concentrate was swapped with isopropanol (3.5 L, 2.5 L/kg) under vacuum at 40° C. to 45° C. twice to about 1.5 L to produce Benzyl (1S,3S)-3-hydroxycyclobutane-1-carboxylate (5a) (1677 g, 95.8 HPLC area % purity, 90.6% assay by HPLC) as a pale brown liquid in 94% yield. A sample was withdrawn, distilled to dryness under reduced pressure (<10 mbar) at 45° C. to 50° C. and the resulting liquid analyzed by NMR and LCMS.

¹H NMR (400 MHz, CDCl₃): 7.33-7.43 (m, 5H), 5.21 (s, 2H), 3.39-3.48 (m, 2H), 3.28-3.34 (m, 3H). ¹³C NMR (75 MHz, CDCl₃): 203.6, 173.9, 135.5, 128.7 (2C), 128.5, 128.3 (2C), 67.1, 51.6 (2C), 27.4. MS: m/z 222.1 (M+H₂O)⁺

### Example 2: Synthesis of Benzyl (1S,3S)-3-hydroxycyclobutane-1-carboxylate (4a)

### Method A: Metal Hydride Catalyst Keto Reduction

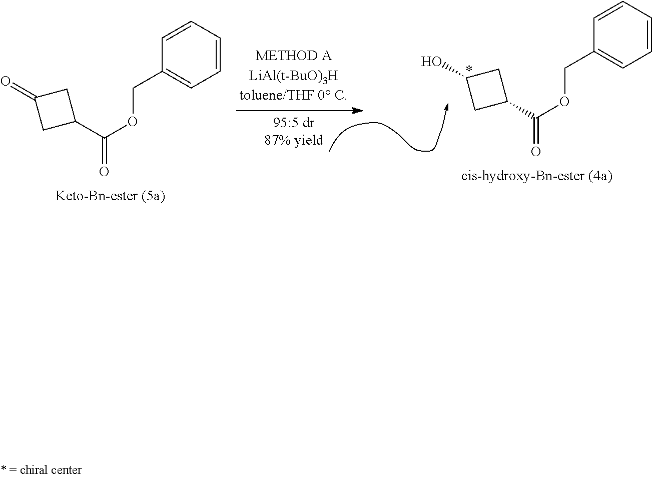 [SMILES: O=C1CC(C(=O)OCc2ccccc2)C1 ; O=C(OCc1ccccc1)[C@H]1C[C@@H](O)C1]

To a pre-cooled (−5° C. to 5° C.) solution of compound (5a) (1.33 kg (75.3% assay by HPLC), 4.90 mol, 1.0 eq.) in THF (10.0 L, 10 L/kg) in a 30 L glass reactor was added a 1 M solution of lithium tri-tert-butoxy aluminum hydride in THF (5.4 L, 5.39 mol, 1.1 eq.) dropwise using a cannula over 2.5 h under nitrogen atmosphere. After the addition of the reagent was complete, the reaction mixture was stirred at −5° C. to 5° C. for another 1 h. After the reaction was adjudged complete (by HPLC), the reaction mixture was cautiously quenched by the addition of aq. 1.5 N HCl (16.0 L, 16 L/kg) and the contents diluted with EtOAc (10.0 L, 10 L/kg). The contents were gradually warmed to 20° C. to 30° C. and stirred at 20° C. to 30° C. for 20 minutes. The aqueous phase was separated and extracted with EtOAc (5.0 L, 5 L/kg). The combined organic phase was washed with 30 wt % aq. NaCl solution (5.0 L, 5 L/kg) and distilled under vacuum at 40-45° C. to ˜2 L. The resulting concentrate was swapped with toluene (2.5 L, 2.5 L/kg) under vacuum at 40° C. to 45° C. twice to about 1.5 L to give compound (4a) (1370 g, 84.7 HPLC area % purity, 65.9% assay by HPLC) as a pale brown liquid in 89% yield.

### Method B: Enzymatic Keto Reduction

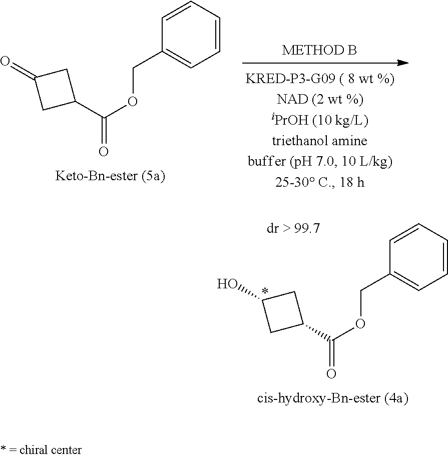 [SMILES: O=C1CC(C(=O)OCc2ccccc2)C1 ; O=C(OCc1ccccc1)[C@H]1C[C@@H](O)C1]

Triethanol amine buffer was prepared by the addition of 188 g of triethanol amine and 1.68 g of MgSO₄ to 13.0 L of demineralized water. pH of the solution was found to be 10.0 and was adjusted to 7.0 by the addition of about 0.8 L of 1.5N aq. HCl.

To a solution of benzyl (1R,3R)-3-bromocyclobutane-1-carboxylate (3a) (1.43 kg (90.6% assay by HPLC), 4.89 mol, 1.0 eq.) in isopropanol (13.0 L, 10 L/kg) in a glass reactor were charged KRED-P3-G09 (104 g, 8 wt %) and NAD (26 g, 2 wt %) under a nitrogen atmosphere at 25° C. to 30° C. Triethanol amine buffer (pH 7.0, 13.0 L, 10 L/kg) was added and the resulting contents stirred at 25° C. to 30° C. for 18 h. After the reaction was adjudged complete by HPLC, the reaction mixture was suction filtered through a short bed of CELITE® and the bed washed with EtOAc (13.0 L, 10 L/kg). The filtrate was distilled under vacuum at 40° C. to 45° C. to remove most of the organic solvent and the resulting solution was diluted with EtOAc (13.0 L, 10 L/kg). The aqueous phase was separated and extracted with EtOAc (13.0 L, 10 L/kg) twice. The combined organic phase was washed with 30 wt % aq. NaCl solution (6.5 L, 5 L/kg) and distilled under vacuum at 40° C. to 45° C. to about 2 L. The resulting concentrate was swapped with toluene (3.3 L, 2.5 L/kg) under vacuum at 40° C. to 45° C. twice to about 1.5 L to give compound (4a) (1592 g, 97.1 HPLC area % purity, 78.4% assay by HPLC) as a brown liquid in 95% yield.

A sample was withdrawn, distilled to dryness under reduced pressure (<10 mbar) at 45° C. to 50° C. and the resulting liquid analyzed by NMR and LCMS. ¹H NMR (400 MHz, CDCl₃): 7.31-7.42 (m, 5H), 5.15 (s, 2H), 4.18 (p, J=7.0 Hz, 1H), 3.21 (bs, 1H), 2.57-2.71 (m, 3H), 2.22-2.27 (m, 2H). ¹³C NMR (75 MHz, CDCl₃): 174.9, 135.9, 128.6 (2C), 128.3, 128.2 (2C), 66.5, 63.1, 37.02, 36.96, 28.9. MS: m/z 207.1 (M+H)⁺

### Example 3: Synthesis of Benzyl (1R,3R)-3-bromocyclobutane-1-carboxylate (3a)

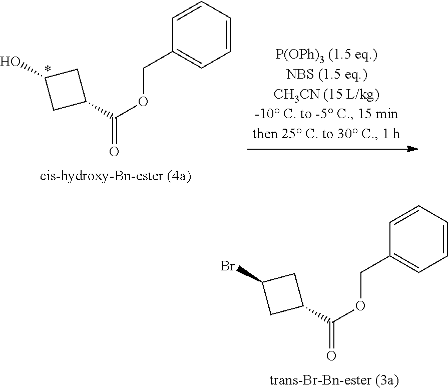 [SMILES: O=C(OCc1ccccc1)[C@H]1C[C@@H](O)C1 ; O=C(OCc1ccccc1)[C@H]1C[C@H](Br)C1]

To a pre-cooled (−5° C. to −10° C.) solution of triphenyl phosphite (226 g, 0.73 mol, 1.5 eq.) in CH₃CN (0.3 L, 3 L/kg) in a glass reactor was added a solution of NBS (131 g, 0.73 mol, 1.5 eq.) in CH₃CN (1.0 L, 10 L/kg) using an addition flask drop wise under a nitrogen atmosphere. It was ensured that the reaction mixture temperature was below 0° C. during the addition. To the resulting solution was added a solution of compound (4a) (128 g (78.4% assay by HPLC), 0.49 mol, 1.0 eq.) in CH₃CN (0.2 L, 2 L/kg) using an addition flask drop wise ensuring the reaction mixture temperature was below 0° C. The reaction was warmed to 25° C. to 30° C. and stirred for 1 h. After the reaction was adjudged complete (by HPLC), the reaction mixture was distilled under vacuum at 25° C. to 30° C. to remove most of the volatiles. The resulting residue was diluted with MTBE (1.0 L, 10 L/kg) and filtered through a short bed of CELITE and the bed washed with MTBE (1.0 L, 10 L/kg). The filtrate was washed with 2 wt % aq. Na₂S₂O₃ (1.0 L, 10 L/kg) twice and distilled under vacuum at 40° C. to 45° C. to about 0.4 L to give crude compound (3a) (414 g, 33.7 HPLC area % purity, 25.5% assay by HPLC) as a dark brown liquid in 81% yield. 3.6 g of (1R,3R)-3-Bromocyclobutane-1-carboxylic crude acid (2a) was also formed as side product in 4% yield.

Analytical data of purified compound (3a): ¹H NMR (400 MHz, CDCl₃): 7.36-7.40 (m, 5H), 5.17 (s, 2H), 4.68 (p, J=7.0 Hz, 1H), 3.46 (septet, J=5.0 Hz, 1H), 2.93-3.00 (m, 2H), 2.69-2.77 (m, 2H). ¹³C NMR (75 MHz, CDCl₃): 174.3, 135.7, 128.6 (2C), 128.3, 128.2 (2C), 66.6, 40.7, 37.6 (2C), 36.0.

Note: The present inventors found that the distillation of the reaction mixture must be controlled at a temperature of under 30° C. Higher temperature was found to result in increased formation of acid (crude 2a-acid).

### Examples 4a and 4b: Synthesis of 2-methyl Propan-2-aminium (1R,3R)-3-bromocyclobutane-1-carboxylic Acid (2a) and Tert-butyl Amine Salt (2a t-BuNH₂ Salt)

### Basic Ester Hydrolysis and Acid Purification

### Example 4a-1: Synthesis of 2-methyl Propan-2-aminium (1R,3R)-3-bromocyclobutane-1-carboxylic Acid (2a)

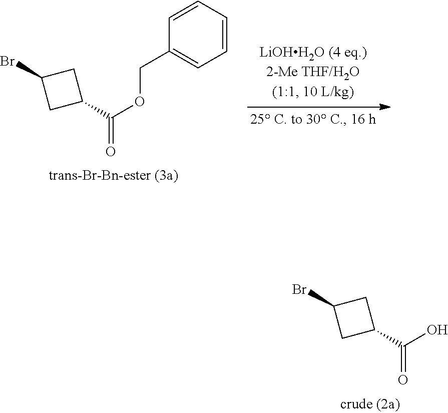 [SMILES: O=C(OCc1ccccc1)[C@H]1C[C@H](Br)C1 ; O=C(O)[C@H]1C[C@H](Br)C1]

To a solution of crude compound (3a) (372 g (25.5% assay by HPLC), 0.35 mol, 1.0 eq.) in 2-methyl-THF (0.48 L, 5 L/kg) was added a solution of LiOH·H₂O (59 g, 1.41 mol, 4.0 eq.) in water (0.48 L, 5 L/kg) at 25° C. to 30° C. The reaction mixture was stirred at 25° C. to 30° C. for 16 h. After the reaction was adjudged complete (by GC), the pH of the reaction mixture was adjusted to 8.0-8.5 by the addition of 1.5 N HCl and diluted with MTBE (0.48 L, 5 L/kg). The phases were separated, and the aqueous phase washed with MTBE (0.48 L, 5 L/kg). pH of the aqueous phase was adjusted to 3.0-3.5 by the addition of 1.5 N HCl and the resulting aqueous phase extracted with MTBE (0.48 L, 5 L/kg) twice. The combined organic extracts were washed with 30 wt % aq. NaCl solution (6.5 L, 5 L/kg) and distilled under vacuum at 40° C. to 45° C. to about 0.2 L. The resulting concentrate was swapped with n-heptane (3.3 L, 2.5 L/kg) under vacuum at 40° C. to 45° C. twice to give crude compound (2a) (330 g, 42.6 GC area % purity, 27.7% assay by GC) as a brown solid in 95% yield.

### Example 4a-2: Synthesis of (1R,3R)-3-bromocyclobutane-1-carboxylate 2-methylpropan-2-aminium

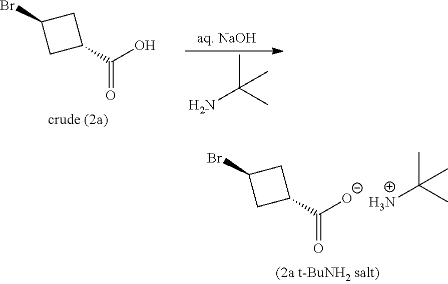

To the crude compound (2a) product of Example 4a was then added n-heptane (0.9 L, 10.0 L/kg) and CELITE (0.33 kg, 100 wt %) and the resulting slurry stirred at 45° C. to 50° C. for 2 h. The hot slurry was suction filtered, and the cake washed with hot n-heptane (0.9 L, 10.0 L/kg). The filtrate was cooled to 45° C. to 50° C., diluted with MTBE (0.2 L, 2 L/kg) and a solution of tert-butyl amine (59 mL, 0.56 mol, 1.1 eq.) in n-heptane (0.2 L, 2 L/kg) was then added under a nitrogen atmosphere at 25° C. to 30° C. and stirred for 16 h. The solids were suction filtered, washed with n-heptane (0.2 L, 2 L/kg) and dried under vacuum at 40° C. to 45° C. to give (1R,3R)-3-bromocyclobutane-1-carboxylate 2-methylpropan-2-aminium (Compound 2a t-BuNH₂ salt) (103 g) as an off-white solid in 96% yield.

¹H NMR (400 MHz, CDCl₃): 6.95 (bs, 3H), 4.68 (p, J=7.0 Hz, 1H), 3.16 (septet, J=4.8 Hz, 1H), 2.81-2.88 (m, 2H), 2.61-2.69 (m, 2H), 1.33 (s, 9H). ¹³C NMR (75 MHz, CDCl₃): 181.0, 50.6, 42.5, 39.0, 38.9 (2C), 27.8 (3C).

### Example 4a-3: Salt Hydrolysis to Compound (2a)

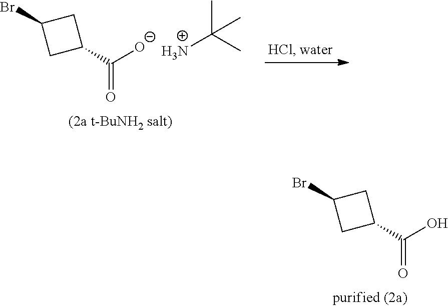

To the pre-cooled (0° C. to 5° C.) solution of the above Compound 2a t-BuNH₂ salt (12.6 g, 50 mmol, 1.0 eq.) in water (50 mL, 4 L/kg) was added 11.2N aq. HCl (5 mL, 0.4 L/kg) drop wise until the pH of the reaction mixture was about 1 to 2. The resulting solids were stirred at 0° C. to 5° C. for 1 h. The solids were then suction filtered, washed with cold water (50 mL, 4 L/kg) and dried under vacuum at 25° C. to 30° C. to give purified compound (2a) (7.4 g) as an off-white solid in 82% yield. ¹H NMR (400 MHz, CDCl₃): 9.33 (bs, 3H), 4.68 (p, J=7.0 Hz, 1H), 3.43 (septet, J=5.0 Hz, 1H), 2.93-3.00 (m, 2H), 2.71-2.78 (m, 2H). ¹³C NMR (75 MHz, CDCl₃):

### Enzymatic Ester Hydrolysis

### Example 4b-1: Synthesis of (1R,3R)-3-bromocyclobutane-1-carboxylic Acid (Crude 2a)

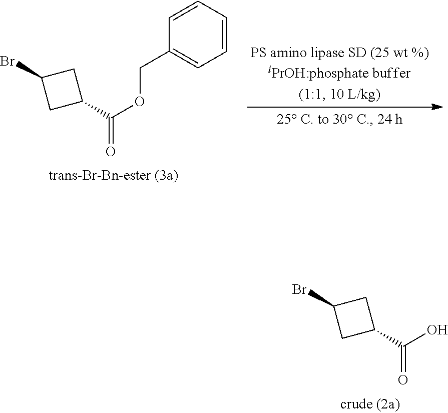 [SMILES: O=C(OCc1ccccc1)[C@H]1C[C@H](Br)C1 ; O=C(O)[C@H]1C[C@H](Br)C1]

To a solution of crude compound (3a) 300 g (25% assay by HPLC), 0.27 mol, 1.0 eq.) in IPA:phosphate buffer (pH 7) (1:1), (3 L, 10 L/kg) was added Lipase PS Amano SD (18.75 g, 0.25 w/w) at 25° C. to 30° C. in a glass reactor. The reaction mixture was stirred at 25° C. to 30° C. for 10 minutes. The pH of the reaction mixture was adjusted from 6.54 to 7.0 using saturated aq. K₃PO₄ solution. The reaction mixture was stirred at 25° C. to 30° C. for 24 h. After the reaction was adjudged complete (by GC), the contents were filtered through CELITE and the CELITE bed was washed with H₂O (3 L, 10 L/kg). The filtrate was distilled in vacuo at 35° C. to remove most of the volatiles. Next, the pH of the residue was adjusted from 6.76 to 3.0 using concentrated HCl. The contents were extracted with MTBE (6 L, 20 L/kg) and the organic phase concentrated completely under vacuum to give crude compound (2a) (266.86 g, 71.4 GC area % purity, 18.0% assay by GC) as a brown solid in 93.8% yield.

### Example 4b-2: Synthesis of 2-methylpropan-2-aminium (1R,3R)-3-bromocyclobutane-1-carboxylate (3)

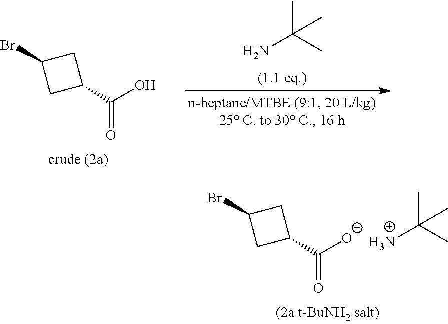

To a solution of crude compound (2a) 252.6 g (46.88 g, assay corrected by HPLC), 0.17 mol, 1.0 eq.) in n-heptane:MTBE (9:1) (940 mL, 20 L/kg) in a glass reactor, was added a solution of tert-butyl amine (21 mL, 0.187 mol, 1.1 eq.) in n-heptane (200 mL, 4.3 L/kg) drop wise using syringe at 25° C. to 30° C. The reaction mixture was stirred under a nitrogen atmosphere at 25° C. to 30° C. for 16 h and observed solids in the reaction mixture. Next, acetone (700 mL, 15 L/kg) was added into the reaction mixture to obtain a uniform slurry. The solids were suction filtered, washed with n-heptane (230 mL, 5 L/kg) and dried under vacuum at 40° C. to 45° C. to give compound (2a t-BuNH₂ salt) (37 g) as an off-white solid in 56% yield.

¹H NMR (400 MHz, CDCl₃): 6.89 (bs, 4H), 4.65 (q, J=0.8 Hz, 1H), 3.14 (septet, J=4.8 Hz, 1H), 2.79-2.85 (m, 2H), 2.58-2.65 (m, 2H), 1.31 (s, 9H).

### Example 4b-3: Synthesis of (1R,3R)-3-bromocyclobutane-1-carboxylic Acid (2a)

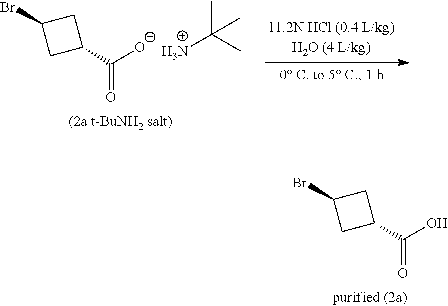

To a solution of compound (2a t-BuNH₂ salt) (35 g, 0.13 mol, 1.0 eq.) in water (140 mL, 4 L/kg) was added 11.2N aq. HCl (19.3 mL, 0.21 mol) at 25° C. to 30° C. drop wise until the pH of the reaction mixture was 1-2. The reaction mixture was warmed to 40-45° C. and the contents stirred at 40° C. to 45° C. for 1 h. Next, the contents were cooled to 25-30° C. and stirred at 25-30° C. for 1 h. Further cooled the contents to 0° C. to 5° C. and stirred at 0° C. to 5° C. for 1 h. The solids were suction filtered, washed with cold water (50 mL, 1.5 L/kg) and dried under vacuum at 25° C. to 30° C. to give purified compound (2a) (12 g) as an off-white solid in 50% yield.

¹H NMR (400 MHz, CDCl₃): 9.33 (bs, 3H), 4.66-4.70 (m, 1H), 4.69 (p, J=7.0 Hz, 1H), 3.43 (septet, J=5.0 Hz, 1H), 2.95-3.01 (m, 2H), 2.72-2.79 (m, 2H). ¹³C NMR (75 MHz, CDCl₃): ¹³C NMR (75 MHz, CDCl₃): 181.1, 40.3, 37.5, 36.0.

### Example 5: Synthesis of (1R,3R)-1-bromo-3-(trifluoromethyl)cyclobutene (5a)

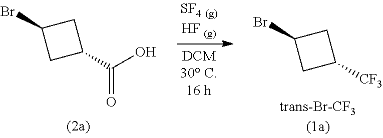 [SMILES: O=C(O)[C@H]1C[C@H](Br)C1 ; FC(F)(F)[C@H]1C[C@H](Br)C1]

To a stirred dichloromethane (DCM) (4.0 vol) solvent was charged trans-1-bromo-3-cyclobutanecarboxylic acid (1.0 eq) and anhydrous hydrogen fluoride (0.13 vol). The solution was transferred to a suitably sized autoclave under static vacuum. Sulphur tetrafluoride (3.0 eq) was charged to the autoclave under 20 Bar of pressure. The reaction was heated at 30° C. for a period of 16 hours, and then allowed to cool back to room temperature. The reaction mixture was quenched on to ice (69.4 eq) and washed through with DCM (13.3 vol). The combined ice/DCM reaction mixture was basified by the addition of 25% potassium hydrogen carbonate solution (13.3 vol). The layers were separated and further extracted with DCM (3×6.7 vol). The combined organic phases were dried with magnesium sulphate and filtered. The product was isolated by distillation (boiling point 112° C. to 114° C.) to give typical yield of 70% to 80% of compound (1a). The yield was 67% at 1000 gram scale. A second distillation was conducted (94% recovery) to ensure quality and purity of the product, which is colorless liquid. GC analysis (excluding DCM) showed 97.4% of the desired trans isomer product (1a) and 1.1% of the cis isomer. GC purity: 96.82%.

¹HNMR (400 MHz, CDCl₃) ppm: 4.60 (quin, J=7.20 Hz, 1H), 3.16-3.30 (m, 1H), 2.84-2.93 (m, 2H), 2.74-2.67 (m, 2H). ¹⁹FNMR (376.46 MHz, CDCl₃): 2.60 (s).

The foregoing invention has been described in some detail by way of illustrations and examples, for purposes of clarity and understanding. Those skilled in the art understand that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited herein are hereby incorporated by reference in their entirety for all purposes to the same extent as if each individual patent, patent application or publication were so individually denoted.

What is claimed is:

1. A stereoselective process for the preparation of a compound having formula (2): 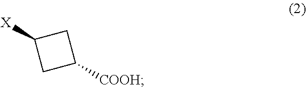
wherein X is halo; comprising
a. contacting a compound of formula (3): 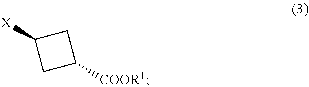
wherein X is as defined above in compound (2); and COOR¹ is an ester group; with an ester hydrolyzing agent in a solvent; to form said compound (2); and optionally followed by a process of purifying said compound (2) by
(a1) contacting said compound (2) with a base in a solvent to form a salt of compound (2); and
(a2) contacting said salt of compound (2) with an acid in a solvent at a low temperature to form a purified form of compound (2).

2. The process according to claim Error! Reference source not found., further comprising preparing said compound (3) comprising:
(b) contacting a compound of formula (4): 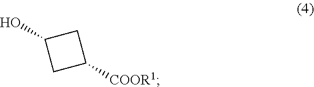
wherein said R¹ is as defined above in compound (3); with a deoxyhalogenating agent in an organic solvent, optionally at a low temperature, to form said compound (3).

3. The process according to claim 2, further comprising preparing said compound (4) comprising: contacting a compound of formula (5): 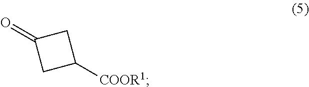
wherein said R¹ is as defined above in compound (4); with
(c1) a metal catalyst in an organic solvent at a low temperature; or
(c2) a biocatalytic agent in an organic solvent and in the presence of a buffer solution; to form said compound (4).

4. The process according to claim 3, further comprising preparing compound (5) comprising:
(d) contacting a compound of formula (6): 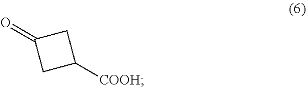
with an R¹ agent, in the presence of a base; wherein R¹ is as defined above in compound (5); in an organic solvent to form said compound (5).

5. The process according to claim 1, further comprising preparing a compound of formula (1): 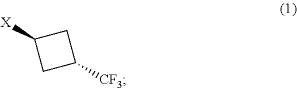
wherein said X is as defined above in compound (2); comprising: contacting said compound (2) with a trifluoromethylating agent in an organic solvent to form said compound of formula (1).

6. The process according to claim 1, wherein X is bromo or iodo.

7. The process according to claim 1, wherein X is bromo.

8. The process according to claim 1, wherein R¹ is (C₁-C₆)alkyl, phenyl, or benzyl.

9. The process according to claim 1, wherein R¹ is benzyl.

10. The process according to claim 1, wherein in (a), said ester hydrolyzing agent is an alkali metal hydroxide or a lipase enzyme.

11. The process according to claim 1, wherein in (a), said ester hydrolyzing agent is an alkali metal hydroxide and said solvent is a mixture of methyl-THF and water.

12. The process according to claim 1, wherein in (a), said ester hydrolyzing agent is sodium hydroxide, potassium hydroxide, or lithium hydroxide.

13. The process according to claim 1, wherein in (a), said ester hydrolyzing agent is lithium hydroxide or sodium hydroxide.

14. The process according to claim 1, wherein in (a), said ester hydrolyzing agent is a lipase enzyme and said solvent is acetone or (C₁-C₆)alcohol.

15. The process according to claim 1, wherein in (a1), said base is a primary, secondary, or tertiary amine base.

16. The process according to claim 1, wherein in (a1), said base is tert-butyl amine and said salt of compound (2) has a formula:

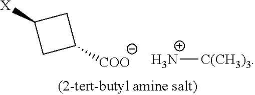

17. The process according to claim 1, wherein in (a1), said solvent is a mixture of n-heptane and MTBE.

18. The process according to claim 1, wherein in (a2), said acid is sulphuric acid, phosphoric acid, or acid halide selected from HCl or HBr.

19. The process according to claim 1, wherein in (a2), said solvent is water.

20. The process according to claim 2, wherein in (b), said deoxyhalogenating agent is triphenyl phosphite in the presence of NBS; or triphenyl phosphine in the presence of NBS.

21. The process according to claim 2, wherein in (b), said organic solvent is DMF, acetonitrile, toluene, or dichloromethane.

22. The process according to claim 3, wherein in (c1), said metal catalyst is a metal hydride.

23. The process according to claim 3, wherein in (c1), said solvent is THF, acetonitrile, toluene, dichloromethane, (C₁-C₈)alcohol, or any mixtures thereof.

24. The process according to claim 3, wherein in (c2), said biocatalytic agent is a ketoreductase enzyme.

25. The process according to claim 3, wherein in (c2), said solvent is (C₁-C₈)alcohol, or a mixture of water and (C₁-C₈)alcohol.

26. The process according to claim 3, wherein in (c2), said buffer is selected from phosphate, triethanol amine, PIPES, BICINE, TES, TRIS, HEPES, TRICINE, CHES, or CAPS.

27. The process according to claim 3, wherein in (c2), said buffer is triethanol amine.

28. The process according to claim 4, wherein in (d), said R¹ agent is (C₁-C₆)alkyl halide, phenyl halide, or benzyl halide.

29. The process according to claim 4, wherein (d) is performed in the presence of a base, wherein said base is a bicarbonate, carbonate, or tri(C₁-C₆)alkylamine base.

30. The process according to claim 4, wherein in (d), the solvent is dichloromethane, DMF, THF, or acetonitrile.

31. The process according to claim 5, wherein said trifluoromethylating agent is sulfur tetrafluoride (SF₄), in the presence of hydrogen fluoride (HF), and optionally in the presence of a solvent.

32. The process according to claim 5, wherein said trifluoromethylating agent is sulfur tetrafluoride (SF₄), in the presence of hydrogen fluoride (HF), and said process is performed in the presence of dichloromethane.
